(19) United States
(12) Patent Application Publication
(10) Pub. No.: US 2009/0118264 A1
(43) Pub. Date: May 7, 2009

(54) Medicament having neovascularization promoting action

(75) Inventors: Ryozo Nagai, Tokyo (JP); Ichiro Manabe, Tokyo (JP); Takayuki Shindo, Tokyo (JP); Hiroshi Iwata, Tokyo (JP); Koichi Shudo, Tokyo (JP); Hiroyuki Kagechika, Tokyo (JP)

Correspondence Address: GREENBLUM & BERNSTEIN, P.L.C., 1950 ROLAND CLARKE PLACE, RESTON, VA, 20191, US

(73) Assignees: Research Foundation Itsuu Laboratory, Tokyo (JP); Ryozo NAGAI, Tokyo (JP); Ichiro MANABE, Tokyo (JP); Takayuki SHINDO, Tokyo (JP); Hiroshi IWATA, Tokyo (JP)

(21) Appl. No.: 12/318,248

(22) Filed: Dec. 23, 2008

Related U.S. Application Data
(62) Division of application No. 11/366,454, filed on Mar. 3, 2006.
(60) Provisional application No. 60/658,175, filed on Mar. 4, 2005.

(51) Int. Cl. A61K 31/551; A61K 31/4164; A61K 31/39; A61K 31/4439; A61P 9/10

(52) U.S. Cl. 514/218; 514/396; 514/434; 514/343

(57) ABSTRACT

Methods of use of medicaments having neovascularization promoting action, which medicaments comprise a retinoid antagonist such as 4-(5H-7,8,9,10-tetrahydro-5,7,7,10,10-pentamethylbenzo[e]naphtho[2,3-b][1-,4]diazepin-13-yl)benzoic acid as an active ingredient.

### CROSS-REFERENCE TO RELATED APPLICATIONS

This application is a divisional of U.S. application Ser. No. 11/366,454, filed Mar. 3, 2006, pending, which claims priority to U.S. Provisional Application No. 60/658,175, filed Mar. 4, 2005. The entire disclosures of each of these applications is incorporated herein by reference.

### TECHNICAL FIELD

The present invention relates to a medicament having a neovascularization promoting action.

### BACKGROUND ART

Surgical therapies such as vascular bypass surgeries or interventional operations such as stent placement have been performed for treatment of diseases in which ischemia is caused in the heart, lower extremities, brain or the like due to vascular pathological conditions such as arteriosclerosis. When these therapies are not operable, however, no alternative effective therapy is available at present. Improvement of the blood flow by promoting neovascularization in an ischemic lesion is believed to become an effective therapy, and a gene therapy for expressing a vascular endothelial growth factor or a hepatocyte growth factor in tissues has been attempted so far. However, problems in efficacy and safety have not yet been solved.

### DISCLOSURE OF THE INVENTION

An object of the present invention is to provide a medicament having a neovascularization promoting action. More specifically, the object of the present invention is to provide a medicament which has a neovascularization promoting action and is used for prophylactic and/or therapeutic treatment of an ischemic disease, or wound or tissue loss such as decubitus, in particular, a medicament useful for prophylactic and/or therapeutic treatment of an ischemic disease resulting from arteriosclerosis (myocardial infarction, angina pectoris, obstructive arteriosclerosis of lower extremities, Buerger's disease, cerebral infarction), decubitus or the like.

The inventors of the present invention conducted various researches to achieve the foregoing object. As a result, they found that retinoid antagonists such as 4-(5H-7,8,9,10-tetrahydro-5,7,7,10,10-pentamethylbenzo[e]naphtho[2,3-b][1-,4]diazepin-13-yl)benzoic acid had actions of proliferating vascular endothelial cells and activating migration, and they enhanced neovascularization. Further, they found that retinoid antagonists had an action of promoting formation of granulation tissues and improving wound healing based on the aforementioned actions. The present invention was accomplished on the basis of the aforementioned findings.

The present invention thus provides a medicament having a neovascularization promoting action, which comprises a retinoid antagonist as an active ingredient. The present invention also provides a medicament for prophylactic and/or therapeutic treatment of an ischemic disease by promoting neovascularization, which comprises a retinoid antagonist as an active ingredient; and a medicament for prophylactic and/or therapeutic treatment of a wound by promoting neovascularization, which comprises a retinoid antagonist as an active ingredient. Examples of the ischemic disease include myocardial infarction, angina pectoris, obstructive, arteriosclerosis of lower extremities, Buerger's disease, cerebral infarction, thoracic outlet syndrome, Takayasu's disease and the like. Examples of the wound include wounds due to physical or chemical damage to tissues, and preferred examples include decubitus.

From another aspect, the present invention provides a method for promoting neovascularization in vivo of a mammal including human, which comprises the step of administering an effective amount of a retinoid antagonist to the mammal including human. The present invention also provides a method for prophylactic and/or therapeutic treatment of an ischemic disease, which comprises the step of administering an effective amount of a retinoid antagonist to a mammal including human; and a method for promoting wound healing, which comprises the step of administering an effective amount of a retinoid antagonist to a mammal including human.

### BRIEF DESCRIPTION OF THE DRAWINGS

FIG. 1 shows results of a neovascularization experiment using a mouse lower extremity ischemia model.

FIG. 2 comprises photographs of angiographic images demonstrating the neovascularization effect of the medicament of the present invention intramuscularly administered to a rabbit lower extremity ischemia model. The blood vessels surrounded by the broken lines are considered to be collateral blood routes. The white arrows indicate the common iliac arteries.

### BEST MODE FOR CARRYING OUT THE INVENTION

In the present specification, the term “retinoid” is used to encompass retinoic acid and compounds having a retinoic acid-like biological activity. Existence of compounds that antagonistically act on a retinoid and attenuate representative actions of the retinoids (typical examples include cell differentiation action, cell growth promoting action, life-sustaining action and the like) is known (Eyrolles, L., et al., Journal of Medicinal Chemistry, 37(10), pp. 1508-1517, 1994). This publication discloses that compounds such as 4-(5H-7,8,9,10-tetrahydro-5,7,7,10,10-pentamethylbenzo[e]naphtho[2,3-b][1,4]diazepin-13-yl)benzoic acid (LE135) act as antagonists of retinoids. Further, the inventors of the present invention found that compounds such as 4-(13H-10,11,12,13-tetrahydro-10,10,13,13,15-pentamethyldinaphtho[2,3-b][1,2-e][1,4]diazepin-7-yl)benzoic acid antagonistically act on a retinoid (Japanese Patent Application No. 7-255912). In the present specification, a substance that antagonistically acts on a retinoid as described above is referred to as a “retinoid antagonist”.

Although it is not intended to be bound by any specific theory, it is considered that a retinoid antagonist binds to a retinoid X receptor (RxR) that forms a dimer with a retinoic acid receptor (RAR) to regulate expression of the physiological activities of a retinoid such as retinoic acid. Whether or not a compound is a retinoid antagonist can be easily determined by those skilled in the art according to the methods described in the aforementioned publications and the like. As a retinoid antagonist which is an active ingredient of the medicament of the present invention, compounds in free forms, or salts, hydrates and solvates thereof may be used. Further, when stereoisomers of the retinoid antagonist exist, arbitrary stereoisomers in pure forms (optically active substance, diastereoisomer, geometrical isomer and the like), or arbitrary mixtures of the stereoisomers, racemates and the like may be used.

Examples of the retinoid antagonist preferably used in the present invention include 4-(5H-7,8,9,10-tetrahydro-5,7,7,10,10-pentamethylbenzo[e]naphtho[2,3-b][1,4]diazepin-13-yl)benzoic acid (LE135);
4-(13H-10,11,12,13-tetrahydro-10,10,13,13,15-pentamethyldinaphtho[2,3-b][1,2-e][1,4]diazepin-7-yl)benzoic acid;
4-[(4-methoxy-3-trichloro[3.3.1.13,7]dec-1-ylbenzoyl)amino]benzoic acid;
4-(5,6,7,8-tetrahydro-5,5,8,8-tetramethyl-1-phenyl-1H-naphtho[2,3-d]imidazol-2-yl)benzoic acid;
4-[[5,6-dihydro-5,5-dimethyl-8-(4-methylphenyl)-2-naphthalenyl]ethynyl]benzoic acid;
2,6-difluoro-4-[[8-bromo-2,2-dimethyl-4-(4-methylphenyl)-6-chromanyl)carbonyl]amino]benzoic acid;
4-[(1E)-2-[7-(heptyloxy)-3,4-dihydro-4,4-dimethyl-1,1-dioxo-2H-1-benzothiopyran-6-yl]-1-propenyl]benzoic acid;
4-[4,5,7,8,9,10-hexahydro-7,7,10,10-tetramethyl-1-(3-pyridylmethyl)anthra[1,2-b]pyrrol-3-yl]benzoic acid, and the like. However, the antagonists are not limited to these examples.

The medicament of the present invention has a neovascularization promoting action and is useful for prophylactic and/or therapeutic treatment of, for example, an ischemic disease and wound. Examples of the ischemic disease include, for example, myocardial infarction, angina pectoris, obstructive arteriosclerosis of lower extremities, Buerger's disease, cerebral infarction, thoracic outlet syndrome, Takayasu's disease and the like. Examples of the wound include wounds resulting from physical or chemical damage to tissues, and more specific examples thereof include decubitus and the like. Examples of the prophylactic treatment of wound include prophylactic treatment of decubitus and the like. However, target diseases of the medicament of the present invention are not limited to these examples. The medicament of the present invention can substantially activate formation of granulation tissues and can improve wounds or tissue loss resulting from physical or chemical damage to tissues.

As the medicament of the present invention, a retinoid antagonist that is an active ingredient per se may be administered. However, a pharmaceutical composition for oral or parenteral administration that can be produced by those skilled in the art according to a known method is desirably prepared and administered. Examples of pharmaceutical compositions suitable for oral administration include tablets, capsules, powders, subtilized granules, granules, solutions, syrups and the like. Examples of pharmaceutical compositions suitable for parenteral administration include injections, suppositories, inhalants, eye drops, nasal drops, ointments, creams, patches and the like. The medicament of the present invention can be preferably parenterally and locally administered to a lesion. Although the mode of the local administration is not particularly limited, it is preferable to locally inject the medicament in the form of injection.

The aforementioned pharmaceutical compositions can be produced by adding pharmacologically and pharmaceutically acceptable additives. Examples of the pharmacologically and pharmaceutically acceptable additives include, for example, excipients, disintegrating agents or disintegrating aids, binders, lubricants, coating agents, dyes, diluents, vehicles, dissolving agents or dissolving aids, isotonic agents, pH modifiers, stabilizers, propellants, tackifiers and the like.

Doses of the medicament of the present invention are not particularly limited and can be suitably selected depending on the type of the active ingredient, potency of the action and the like. Further, the doses can be suitably adjusted depending on various factors that should be usually taken into account, such as the body weight and age of a patient, the type and symptoms of a disease, and administration route. When the medicament of the present invention is locally administered, the medicament can be usually used in an amount in the range of about 0.01 to 1,000 mg per day for adults.

### EXAMPLES

The present invention will be explained more specifically with reference to the following examples. However, the scope of the present invention is not limited to these examples. In the following examples, 4-(5H-7,8,9,10-tetrahydro-5,7,7,10,10-pentamethylbenzo[e]naphtho[2,3-b][1,4]diazepin-13-yl)benzoic acid (LE135) was used as a retinoid antagonist.

### Example 1

### Effect on Neovascularization or Angiogenesis

The effect of LE135 on neovascularization was investigated by using a co-culture system of human umbilical vein endothelial cells and human skin fibroblasts (Neovascularization Kit, Kurabo Industries Ltd.). The vascular endothelial growth factor (VEGF) having a potent neovascularization action, added to 8 wells at a final concentration of 10 ng/ml, was used as positive control. Culture medium alone added to 8 wells without addition of the medicament was used as negative control. LE135 was added to the wells (8 wells for each group) at various concentrations, and culture was performed for 11 days. Immunostaining was performed by using anti-CD31 (PECAM1) antibodies directed to the vascular endothelial cells to determine the areas of luminal structures similar to blood vessels showing positive results and the numbers of branchings as an index of maturity of the formed luminal structures. The luminal structure areas and the numbers of branchings observed in the wells added with the medicament (LE135 or VEGF as the positive control) at various concentrations are shown in Table 1 in terms of relative values based on the luminal structure area and the number of branchings observed in the medicament-free wells as the negative control, which were taken as 100, respectively. It was demonstrated that LE135 significantly promoted neovascularization at concentrations of 10⁻¹⁰ and 10⁻⁹ M.

TABLE 1

| | Area | Number of branching |
|---|---|---|
| Negative control | 100 ± 5.76 | 100 ± 10.80 |
| LE135 10⁻¹¹ M | 109.09 ± 4.89 | 118.70 ± 9.27 |
| LE135 10⁻¹⁰ M | 116.30 ± 5.69 * | 137.40 ± 9.48 * |
| LE135 10⁻⁹ M | 112.52 ± 4.68 | 150.31 ± 10.26 * |
| VEGF (positive control) | 134.88 ± 4.42 | 154.20 ± 7.12 |

The numerical values indicate the means ± standard deviations, and * indicates that there was a significant difference compared with the negative control.

### Example 2

### Neovascularization Experiment Using Mouse Lower Extremity Ischemia Model

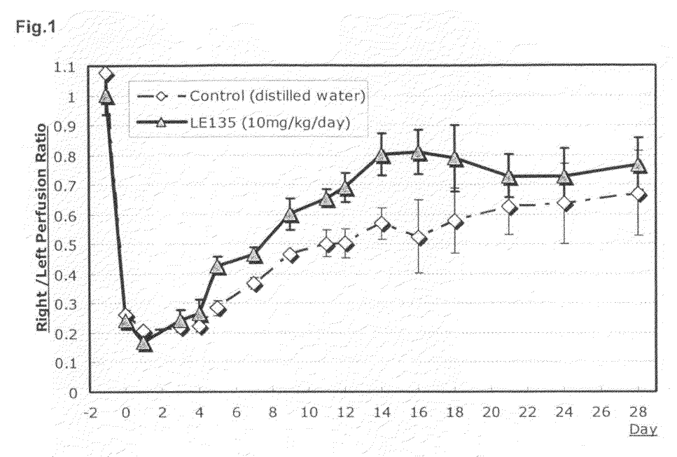

The femoral arteries on one side of 24 to 36-week old wild-type male mice (C3H/HeNCrj) were ligated and separated to prepare lower extremity ischemia model mice. The mice were divided into two groups, i.e., mice orally administered with 5 mg/kg of LE135 (15 animals) and mice not administered with the medicament (15 animals). The blood flow ratio between the affected extremities and the normal extremities was determined by the laser Doppler method on the day before the operation and 1, 3, 4, 5, 7, 9, 12, 14, 16, 18, 21, 24 and 28 days after the operation. The results are shown in FIG. 1. Significant improvement of the blood flow was observed in the LE135-administered mice compared with the no medicament-administered mice. Further, whereas necrosis or amputation was observed in about 30% of the lower extremities of the no medicament-administered mice on the 28th day after the operation, no mouse among the LE135-administered mice was observed to have necrosis or amputation in the lower extremities. In order to detect vascular endothelial cells in muscle tissues of the affected extremities on 21st day after the operation, immunostaining was performed by using anti-CD31 (PECAM1) antibodies. As a result, significantly higher numbers of endothelial cells (CD31-positive cells) were observed in the LE135-administered group compared with the no medicament-administered group (Table 2).

TABLE 2

| | Mice administered with | No medicament- |
|---|---|---|
| | 10 mg/kg of LE135 | administered mice |
| CD31-positive cell | 21.63 ± 4.12 | 13.76 ± 5.42 |
| count per field | | |
| (200×) | | |

### Example 3

### Intramuscular Administration to Rabbit Lower Extremity Ischemia Model

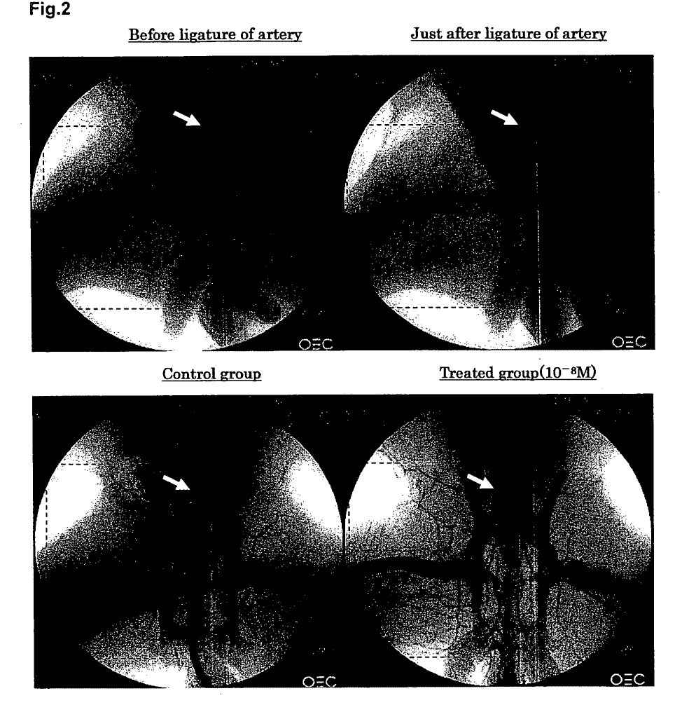

The external iliac arteries on one side of rabbits were ligated and separated, and a 10⁻⁸ M LE135 solution was injected into the femoral muscles on the same side to examine the neovascularization effect of the medicament. A solution of an organic solvent alone in the same amount as used for dissolution of LE135 was used as a negative control. The solutions were administered 3 times per week from the next day of the ligature. Observation of the lower extremities was performed by angiography on the 28th day from the ligature, and evidently better formation of collateral blood routes was observed in the LE135-administered group compared with the control group. The findings of the angiography are shown in FIG. 2. The blood vessels surrounded by the broken lines are considered to be collateral blood routes. In the figure, the white arrows* indicate the common iliac arteries.

### Example 4

### Effect on Neovascularization and Angiogenesis

The effects of RAR antagonists other than LE135 on neovascularization were examined by using a co-culture system of human umbilical vein endothelial cells and human skin fibroblasts (Neovascularization Kit, Kurabo Industries Ltd.). The vascular endothelial growth factor (VEGF) having a potent neovascularization action added to wells at a final concentration of 10 μg/ml was used as positive control. Culture medium alone added to wells without addition of the medicament was used as negative control. Each compound was added at a concentration of 10⁻⁹ mol/L, and culture was performed for 11 days. Immunostaining was performed by using anti-CD31 (PECAM1) antibodies directed to vascular endothelial cells to determine the areas of luminal structures similar to blood vessels showing positive results and the numbers of branchings as an index of maturity of the formed luminal structures. The luminal structure areas and the numbers of branchings observed in the wells added with the medicament at each given concentration are shown in the following table in terms of relative values based on the luminal structure area and the number of branchings observed in the medicament-free wells as the negative control, which were taken as 100, respectively. LE540 and BIBn significantly increased the blood vessel luminal structure area.

TABLE 3

| | Area | Number of branchings |
|---|---|---|
| Negative control | 100.00 ± 5.34 | 100.00 ± 6.80 |
| LE135 | 158.73 ± 6.89 * | 170.09 ± 14.87 |
| LE540 | 172.02 ± 22.00 * | 195.33 ± 24.49 * |
| TD550 | 101.96 ± 10.08 | 131.78 ± 12.10 |
| BIBn | 148.47 ± 22.13 * | 150.31 ± 5.13 |
| VEGF (positive control) | 202.52 ± 18.40 * | 321.50 ± 42.17 * |

The numerical values indicate the means ± standard deviations, and * indicates that there was a significant difference compared with the negative control.

### INDUSTRIAL APPLICABILITY

The medicament of the present invention has a neovascularization promoting action and is useful as a medicament for prophylactic and/or therapeutic treatment of ischemic diseases and wounds on the basis of the action.

What is claimed is:

1. A method for promoting neovascularization in vivo in a mammal, comprising administering an effective amount of a retinoid antagonist to a mammal, wherein the retinoid antagonist is a compound selected from: 4-(5H-7,8,9,10-tetrahydro-5,7,7,10,10-pentamethylbenzo[e]naphtho[2,3-b][1,4]diazepin-13-yl)benzoic acid;
4-(13H-10,11,12,13-tetrahydro-10,10,13,13,15-pentamethyldinaphtho[2,3-b][1,2-e][1,4]diazepin-7-yl)benzoic acid;
4-[(4-methoxy-3-trichloro[3.3.1.13,7]dec-1-ylbenzoyl)amino]benzoic acid;
4-(5,6,7,8-tetrahydro-5,5,8,8-tetramethyl-1-phenyl-1H-naphtho[2,3-d]imidazol-2-yl)benzoic acid;
4-[[5,6-dihydro-5,5-dimethyl-8-(4-methylphenyl)-2-naphthalenyl]ethynyl]benzoic acid;
2,6-difluoro-4-[[8-bromo-2,2-dimethyl-4-(4-methylphenyl)-6-chromanyl)carbonyl]amino]benzoic acid;
4-[(1E)-2-[7-(heptyloxy)-3,4-dihydro-4,4-dimethyl-1,1-dioxo-2H-1-benzothiopyran-6-yl]-1-propenyl]benzoic acid;
4-[4,5,7,8,9,10-hexahydro-7,7,10,10-tetramethyl-1-(3-pyridylmethyl)anthra[1,2-b]pyrrol-3-yl]benzoic acid; and
physiologically acceptable salts thereof.

2. A method for promoting neovascularization in vivo in a mammal for prophylactic and/or therapeutic treatment of an ischemic disease, comprising administering an effective amount of a retinoid antagonist to a mammal, wherein the retinoid antagonist is a compound selected from:
4-(5H-7,8,9,10-tetrahydro-5,7,7,10,10-pentamethylbenzo[e]naphtho[2,3-b][1,4]diazepin-13-yl)benzoic acid;
4-(13H-10,11,12,13-tetrahydro-10,10,13,13,15-pentamethyldinaphtho[2,3-b][1,2-e][1,4]diazepin-7-yl)benzoic acid;
4-[(4-methoxy-3-trichloro[3.3.1.13,7]dec-1-ylbenzoyl)amino]benzoic acid; 4-(5,6,7,8-tetrahydro-5,5,8,8-tetramethyl-1-phenyl-1H-naphtho[2,3-d]imidazol-2-yl)benzoic acid;
4-[[5,6-dihydro-5,5-dimethyl-8-(4-methylphenyl)-2-naphthalenyl]ethynyl]benzoic acid;
2,6-difluoro-4-[[8-bromo-2,2-dimethyl-4-(4-methylphenyl)-6-chromanyl)carbonyl]amino]benzoic acid;
4-[(1E)-2-[7-(heptyloxy)-3,4-dihydro-4,4-dimethyl-1,1-dioxo-2H-1-benzothiopyran-6-yl]-1-propenyl]benzoic acid;
4-[4,5,7,8,9,10-hexahydro-7,7,10,10-tetramethyl-1-(3-pyridylmethyl)anthra[1,2-b]pyrrol-3-yl]benzoic acid;
and physiologically acceptable salts thereof.

3. The method according to claim 2, wherein the ischemic disease is myocardial infarction, angina pectoris, obstructive arteriosclerosis of lower extremities, Buerger's disease, cerebral infarction, thoracic outlet syndrome, or Takayasu's disease.

4. A method for prophylactic and/or therapeutic treatment of a wound by promoting neovascularization, comprising administering an effective amount of a retinoid antagonist to a mammal, wherein the retinoid antagonist is a compound selected from:
4-(5H-7,8,9,10-tetrahydro-5,7,7,10,10-pentamethylbenzo[e]naphtho[2,3-b][1,4]diazepin-13-yl)benzoic acid;
4-(13H-10,11,12,13-tetrahydro-10,10,13,13,15-pentamethyldinaphtho[2,3-b][1,2-e][1,4]diazepin-7-yl)benzoic acid;
4-[(4-methoxy-3-trichloro[3.3.1.13,7]dec-1-ylbenzoyl)amino]benzoic acid; 4-(5,6,7,8-tetrahydro-5,5,8,8-tetramethyl-1-phenyl-1H-naphtho[2,3-d]imidazol-2-yl)benzoic acid;
4-[[5,6-dihydro-5,5-dimethyl-8-(4-methylphenyl)-2-naphthalenyl]ethynyl]benzoic acid;
2,6-difluoro-4-[[8-bromo-2,2-dimethyl-4-(4-methylphenyl)-6-chromanyl)carbonyl]amino]benzoic acid; 4-[(1E)-2-[7-(heptyloxy)-3,4-dihydro-4,4-dimethyl-1,1-dioxo-2H-1-benzothiopyran-6-yl]-1-propenyl]benzoic acid;
4-[4,5,7,8,9,10-hexahydro-7,7,10,10-tetramethyl-1-(3-pyridylmethyl)anthra[1,2-b]pyrrol-3-yl]benzoic acid; and
physiologically acceptable salts thereof.

5. The method according to claim 1, wherein the mammal is a human.

6. The method according to claim 2, wherein the mammal is a human.

7. The method according to claim 4, wherein the mammal is a human.
